(19) United States
(12) Patent Application Publication
(10) Pub. No.: US 2007/0196518 A1
(43) Pub. Date: Aug. 23, 2007

(54) Composition and Method for Mitigating a Negative Effect of Alcohol Consumption

(76) Inventors: Karol Wojewnik, Toronto (CA); Alicia Wojewnik, Toronto (CA); Peter Wojewnik, Toronto (CA)

Correspondence Address: SUZANNAH K. SUNDBY, SMITH, GAMBRELL & RUSSEL, LLP, 1850 M STREET NW #800, WASHINGTON, DC, 20036, US

(21) Appl. No.: 11/566,334

(22) Filed: Dec. 4, 2006

Related U.S. Application Data
(60) Provisional application No. 60/750,350, filed on Dec. 15, 2005.

(51) Int. Cl. A61K 36/82; A61K 36/906

(52) U.S. Cl. 424/729; 424/756

(57) ABSTRACT

A composition for the prevention and treatment of symptoms (for example, a hangover) associated with ethyl alcohol consumption. The composition may comprise a gingerol, a silymarin, a catechin, and a group B vitamin. Treatment of symptoms associated with the consumption of ethyl alcohol may comprise administration of the composition prior to, during, or after the consumption of the alcohol.

### CROSS REFERENCE TO RELATED APPLICATIONS

This application claims the benefit of U.S. Provisional Patent Application Ser. No. 60/750,350, filed 15 Dec. 2005, which is herein incorporated by reference in its entirety.

### FIELD OF INVENTION

The present invention relates to mitigation of a negative effect of alcohol consumption. More particularly, the present invention relates to a composition that may be administered prior to, during, or after consuming alcohol.

### BACKGROUND OF THE INVENTION

Consumption of ethyl alcohol may be followed by a syndrome known as hangover. Symptoms of a hangover include headache, dehydration, nausea, nerve and muscle pain, lethargy, congestion, chills, tremor, diarrhea and fever. These symptoms may be particularly severe, especially after heavy consumption of alcoholic drinks. While not life-threatening, these symptoms are unpleasant and may interfere with job performance or home life. Additionally, the condition of alcohol-induced hangover may cause a person to suffer a social stigma associated with the condition. Alcohol also causes a number of long-term negative effects in the human organism. Although every organ system in the body is negatively affected by alcohol, the organs which are typically vulnerable are the nervous system, the liver, and the skin.

The symptoms of hangover have been treated with painkillers, such as aspirin or non-steroid anti-inflammatory drugs (“NSAIDs”) or antacids. However, painkillers may cause additional stomach upset and do not, alone, treat other symptoms of hangover. Acetaminophen, which is neither aspirin nor an NSAID, has been used to treat headaches and pains associated with hangover, but acetaminophen in combination with ethanol can result in extensive liver damage. The diuretic effect of alcohol results in dehydration and is generally treated with ingestion of large amounts of water after drinking ethanol. The ingestion of water after drinking alcohol, however, does not alleviate negative effects other than dehydration, for example the problems associated with the build up of acetaldehyde in the liver.

Other means for treating hangover symptoms include additional ethyl alcohol consumption for its analgesic properties. Such remedies ultimately prolong the effects of overindulgence, are dangerous and may lead to alcohol addiction.

Herbal remedies have been used to alleviate or prevent symptoms of hangover. Examples of herbal remedies include teas made from the extracts of leaves, stems or roots of alder or mountain ash. This tea is rich in tannin that is said to provide protection to the stomach mucosa. Extracts of fruits of other plants may be added to the teas for their Vitamin C, amino acids and beta-carotene content. The beta-carotene is effective at clearing up cough and phlegm.

Ginkgo biloba extract and taurine are components of another composition that has been used to alleviate hangover symptoms. The taurine is taken for liver protection and the Ginkgo biloba for its antioxidant effects against ethanol-derived oxidation and also facilitates circulation in the brain.

Several methods of preventing alcohol absorption or delaying drunkenness are known. Abstinence, charcoal ingestion and charcoal ingestion in combination with Vitamin B-6 and Ephedra are examples. Abstinence is not a practical solution for many people. Charcoal ingestion is intended to absorb the alcohol in the stomach or small intestines resulting in a lower blood alcohol level. However, charcoal also absorbs important nutrients, and therefore frequent use may result in nutritional deficiencies.

Several patent publications disclose hangover remedies. For example, U.S. Pat. No. 6,913,769 filed Feb. 18, 2003, U.S. Pat. No. 6,312,736 filed Dec. 9, 1999, U.S. Pat. No. 4,496,548 filed Feb. 4, 1983, Japanese Patent no. 6263648 published Sep. 20, 1994, and French Patent No. 2861739 published May 6, 2005 provide compositions for protecting against negative effects of alcohol consumption.

Despite available remedies that are thought to protect against negative effects of alcohol consumption, approximately 20 million people suffer from a hangover every month and hangovers are estimated to result in 148 billion dollars in lost work every year, in the United States alone. Even further productivity is lost when considering the long-term negative effects of alcohol consumption.

Accordingly, there is a need for compositions and protective treatments for mitigating a negative effect of alcohol consumption.

### SUMMARY OF THE INVENTION

The present invention relates to mitigation of a negative effect of alcohol consumption. More particularly, the present invention relates to a composition that may be administered prior to, during, or after consuming alcohol.

Without wishing to be limiting, it is an object of the invention to provide an improved composition and method for mitigating a negative effect of alcohol consumption, while allowing for enjoyment of the desired social effects of consuming alcohol.

According to the present invention there is provided a method of preventing, treating or both preventing and treating a negative effect of alcohol consumption in a person in need of such treatment, which comprises the administration to such person of a composition comprising a gingerol, a silymarin, a catechin and a group B vitamin.

Also provided by the method of the present invention as described above, the composition may be administered orally prior to consuming alcohol or when the negative effect is observed. Further, the composition may be administered orally prior to alcohol consumption, during alcohol consumption, after alcohol consumption, or any combination thereof.

The present invention also provides a composition for administration to a subject to mitigate a negative effect of alcohol consumption, the composition comprising a gingerol, a silymarin, a catechin, and a group B vitamin. Preferably the gingerol is from Zingiber officinale. Preferably, the silymarin is from Silybum marianum fructus. Preferably the catechin is epigallocatechin gallate from Camellia sinensis folium. Preferably, the group B vitamin is two or more of vitamin B1, vitamin B6, and vitamin B12, more preferably vitamin B1, vitamin B6 and vitamin B12.

The present invention also contemplates a composition as provided above formulated as an oral dosage form, preferably a liquid tincture, beverage, food product, lozenge, tablet, caplet, or capsule.

In an embodiment of the present invention, there is provided a composition comprising:
Gingerol in an amount of about 0.5% to about 78% (w/w), for example, but not limited to 0.5%, 1%, 5%, 10%, 20%, 25%, 30%, 35%, 40%, 45%, 50%, 55%, 60%, 65%, 70%, 75% or 78% (w/w). Further, the amount of gingerol present may be defined by a range of any two of the values listed above.
Silymarin in an amount of about 0.6% to about 96% (w/w), for example, but not limited to 0.6%, 1%, 2%, 5%, 10%, 20%, 25%, 30%, 40%, 50%, 60%, 70%, 80%, 90%, 95% or 96% (w/w). Further, the amount of silymarin present may be defined by a range of any two of the values listed above.
EGCG in an amount of about 1% to about 98% (w/w), for example, but not limited to 1%, 2%, 5%, 10%, 20%, 25%, 30%, 40%, 50%, 60%, 70%, 80%, 90%, 95%, 96%, 97%, or 98% (w/w). Further, the amount of EGCG present may be defined by a range of any two of the values listed above.
Vitamin B1 in an amount of about 0.04% to about 88% (w/w), for example, but not limited to 0.04%, 0.1%, 0.5%, 1%, 2%, 5%, 10%, 20%, 25%, 30%, 40%, 50%, 60%, 70%, 80%, 85%, 87%, or 88% (w/w). Further the amount of vitamin B1 present may be defined by a range of any two of the values listed above.
Vitamin B6 in an amount of about 0.06% to 88% (w/w), for example, but not limited to 0.06%, 0.1%, 0.5%, 1%, 2%, 5%, 10%, 15%, 20%, 25%, 30%, 40%, 50%, 60%, 70%, 80%, 85%, 87%, or 88%. Further, the amount may be defined by a range of any two of the values listed above.
Vitamin B12 in an amount of about 7×10⁻⁷% to 12% (w/w), for example, but not limited to 7×10⁻⁶%, 7×10⁻⁵%, 7×10⁻⁴%, 7×10³%, 7×10⁻²%, 7×10⁻¹%, 1%, 2%, 5%, 6%, 7%, 8%, 9%, 10%, 11% or 12%. Further the amount may be defined by a range of any two of the values listed above.

The present invention also contemplates a composition comprising:
gingerol in an amount of about 0.8% (w/w);
Silymarin in an amount of about 23% (w/w);
EGCG in an amount of about 19% (w/w);
vitamin B1 in an amount of about 16% (w/w);
vitamin B6 in an amount of about 40% (w/w) and;
vitamin B 12 (Cyanocobalamin) in an amount of about 0.1% (w/w).

In a further embodiment of the present invention as described above, the composition comprises:

| | | |
|---|---|---|
| EGCG (from Camellia sinensis folium) | 60 | mg |
| Silymain (from Silybum marianum fructus) | 72 | mg |
| Gingerols (from Singiber officinale radix) | 2.5 | mg |
| Vitamin B1 (thiamine) | 50 | mg |
| Vitamin B6 (pyridoxine) | 125 | mg |
| Vitamin B12 (cyanocobalamin) | 400 | mcg. |

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a representative processing flow chart for ginger.

### DETAILED DESCRIPTION

The present invention relates to mitigation of a negative effect of alcohol consumption. More particularly, the present invention relates to a composition that may be administered prior to, during, or after consuming alcohol.

Passages throughout the disclosure providing details in regard of any cause and effect, mechanism of action or biochemical pathway or the like are non-exhaustive, not meant to be bound by theory or limiting in any manner.

The following description is of a preferred embodiment.

Most adults have experienced an alcohol-induced hangover. Depending on severity of the hangover symptoms can include, without limitation, nausea, vomiting, and diarrhea, dehydration, irritability, headache, loss of appetite, sleeplessness, respiratory difficulties, tremors of hands or limbs, dizziness, hypersensitivity to light and noise, soreness or weakness of joints, muscle aches, inability to concentrate and solve problems, weakness, fatigue, and lethargy.

Alcohol consumption may also lead to organ damage. Long-term alcohol drinkers have been known to suffer from liver damage such as fatty liver, alcoholic hepatitis, or alcoholic cirrhosis. Other types of alcohol-induced organ damage include chronic gastritis, damage to the esophagus, pancreatitis, brain damage such as alcoholic dementia, damage to the heart and the circulatory system such as high blood pressure and hemorrhagic stroke, muscle weakness, exacerbation of skin conditions such as psoriasis and eczema, loss of libido and potency, shrinking of sexual organs such as breasts and ovaries in females and testicles and penis in males, reduced fertility, loss of pubic hair and if cirrhosis is present, increased breast size and loss of body hair in males, and alcohol-related cancers such as those of the mouth, esophagus, liver, stomach, colon, rectum, and breast cancer in women.

Alcohol consumption may also cause a socially embarrassing state of drunkenness. The euphoria of alcohol intoxication may be accompanied by mental and motor impairment, for example, without limitation, slurring of words, reduced short term memory, loss of balance, and other attributes of an alcohol-induced state commonly referred to as being “tipsy”. Many professionals, such as businessman are expected to drink alcohol while maintaining proper speech and motor coordination.

Certain aspects of the present invention provide methods, uses, compositions or a combination thereof, for mitigating a negative effect of alcohol consumption. Without wishing to be limiting in any manner, the negative effect may be associated with alcohol-induced drunkenness, hangover, organ damage, or any combination thereof.

The terms “ethyl alcohol” and “ethanol” as used herein refer to any ethyl alcohol containing or alcoholic beverage including beer, wine, spirits and the like. Ethyl alcohol, or ethanol, is typically consumed by ingesting a liquid containing ethyl alcohol. It is typically produced by yeast in a fermentation process which converts sugars to alcohol and is then consumed, as is, in the case of beer or wine, for example. The fermented alcohol may be distilled to form spirits and then consumed, as is, in the case of vodka or whiskey, for example. Additional sweeteners, water, color and other ingredients are sometimes added.

Once consumed, the ethyl alcohol in the beverage is absorbed by the stomach or small intestine and transferred to the liver through blood vessels. Without wishing to be bound by theory or limiting in any manner, metabolism of the alcohol takes place in a two-step enzymatic reaction. First, the alcohol is oxidized to acetaldehyde by alcohol dehydrogenase. Second, the acetaldehyde is oxidized to acetic acid by acetaldehyde dehydrogenase. The rate at which alcohol is metabolized depends upon the amount and activity of these enzymes. The activity largely depends on the level of NADH (nicotinamide adenine dinucleotide hydrogen) in the body. Excessive NADH produced by these reactions inhibits the activity of the enzymes. The rate at which alcohol is metabolized and eliminated from the body varies greatly among individuals. This is likely due to different levels of relevant enzymes or different rates of enzymatic activity.

The negative effects of consumption of ethanol may be due to the toxic effects of acetaldehyde, hypoglycemia (low blood sugar level), free radical formation, inflammation, liver damage, dehydration, low levels of vital nutrients, excessive levels of NADH in the body, low levels of reduced glutathione, increased production of lactic acid and the consequent drop in blood pH level, impurities in alcoholic beverages or any combination thereof

Acetaldehyde provokes disturbances in bodily processes by, for example, forming adducts with hemoglobin and proteins in plasma, and inhibiting the transfer of reducing agents along the mitochondrial respiratory chain. Acetaldehyde also accumulates in the cerebellum causing headache by contracting cerebral blood vessels thereby decreasing blood flow resulting in pain.

Metabolizing alcohol to acetate drives the formation of NADH. This is a high energy compound that is transferred to the mitochondria for energy production. Excessive NADH molecules inhibit multiple steps along the glycolysis, gluconeogenesis, and the tricarboxylic acid cycle pathways. Whereas the synthesis of fatty acids is stimulated. This leads to hypoglycemia (a low blood glucose level). Since the brain depends mainly on glucose as an energy source, a low level of glucose could be one of the causes of hangover.

The excessive NADH also diverts pyruvate (an end product of glycolysis) into the production of lactic acid. This decreases the pH of blood, which in turn interferes with multiple biochemical reactions. It may also cause protein denaturation. Such deleterious effects slow down the metabolism of alcohol, and thus potentiate its negative effects including hangover. The conversion of pyruvate into lactate, also decreases the available pyruvate necessary for gluconeogenesis, thus potentiating the hypoglycemia incurred by alcohol ingestion. Furthermore, the excessive NADH drives fatty acid synthesis by stimulating the conversion of glyceraldehyde-3-P into glycerol-3-P. This process leads to the fatty liver syndrome and ultimately liver cirrhosis. It is interesting to note that the most common vitamin deficiencies during alcohol intake are the same vitamins which play important roles in these pathways. These are the vitamins, B1, B6, B12.

Vitamins B1 and B12 in combination prevent the slowing down of these biochemical pathways in cases of deficiency. They also prevent the most common neurological lesions found in alcoholics. Vitamin B6 is involved in detaching glucose molecules from glycogen for its transfer into the blood.

Negative effects of alcohol consumption may also be due to oxidative stress. Alcohol is a toxin and as such it can cause organ inflammation and free radical formation. Neutralizing inflammation and free radicals could mitigate a major component of the cause of a hangover as well as organ damage. Silymarin from Milk Thistle, Gingerol from Ginger, Vitamin B1, or a catechin from green tea are non-limiting examples of compounds that may play a part in neutralizing inflammation.

Furthermore, compounds such as gingerols, silymarins, group B vitamins, catechins, or functional equivalents thereof, may inhibit the inflammatory process and scavenge free radicals by different means, and therefore a combination of such compounds may be useful. For example, epigallocatechin (EGCG) may promote monocytic apoptosis thus potentiating the anti-inflammatory reaction. Monocytes are cells which among other cells can cause inflammation. Gingerol from the Zingiber officinale plant may inhibit lipoxygenase, cyclooxygenase, and thromboxane synthase enzymes. These are crucial enzymes in the formation of leukotrenes, prostaglandins, and thromboxane respectively; compounds which can cause inflammation. Vitamin B1 plays a crucial part in the pentose phosphate pathway. This pathway generates NADPH, the compound used to regenerate reduced glutathione. Reduced glutathione in turn is a powerful system for scavenging free radicals. Free radicals are compounds which damage cell membranes, thus ultimately causing cell aging and cell death. Silymarin from Milk Thistle may decrease mitochondrial glutathione oxidation, thus increasing the reduced glutathione needed to neutralize free radicals. It also inhibits leukotriene formation by Kupffer cells (monocytes located in the liver). Thus, without wishing to be bound by theory, providing compounds that act at one or more of these pathways could mitigate hangover or organ damage effects incurred by inflammation and the free radical formation during alcohol intake.

One or more of a silymarin, a catechin, a gingerol, a group B vitamin are used for mitigating negtive effects of alcohol consumption in certain examples of the present invention.

Silymarin.

Silymarin is a compound contained in a plant named Milk thistle (Silybum marianum) and is commonly known as St. Mary's thistle and Our Lady's thistle. Silymarin comprises various forms of hepatoprotectant flavonolignins (compounds that protect the liver). Silymarins protect the liver against a variety of toxins. The active components in silymarin include silybin, silychristin and silydianin. Silymarin repair mechanisms include increased synthesis of cellular proteins, increased rate of hepatocellular repair and anti-oxidant activity. Silymarin contributes to the regenerative activity of the liver by stimulating RNA polymerase and increasing synthesis of ribosomes. The flavonoids in Silymarin are free radical scavengers and stabilizers of plasma membrane. The free radicals produced during alcohol ingestion damage cells and organ systems and thus contribute to the feeling of hangover. Silymarin in this case functions as an anti-oxidant. It also inhibits lipid peroxidation of hepatocyte (liver cell) membrane, thus protecting the liver. Perhaps one of its most important functions is increasing the level of reduced glutathione (glutathione is necessary to scavenge free radicals). Its other important protective functions are: decreased fibrinogenesis in the liver, increased production of hepatoprotective salts (beta muricholate and ursodeoxycholic acid), inhibition of leukotriene formation by Kupffer cells, as well as altering the structure of the outer membrane of hepatocytes as to prevent penetration of toxins.

Catechins.

Catechins are bioflavonoids, polyphenols and anti-oxidants. Sources of catechins include, without limitation, dark chocolate, apples, white tea, and green tea. Catechins have been shown to enhance immune system function.

Tea contains four main catechin substances: EC, ECG, EGC and EGCG. Without wishing to be considered limiting, epigallocatechin gallate (EGCG) is thought to be the most powerful of these catechins.

Epigallocatechin Gallate (EGCG).

This compound has a significant anti-oxidant effect as well as a stimulatory effect. It prevents early alcohol-induced liver injury most likely by preventing oxidative stress. It also promotes monocytic apoptosis thus decreasing the inflammatory reaction. By its stimulatory action it increases the metabolic rate. Increased metabolic rate means increased ATP formation in the oxidative respiratory chain of the mitochondrion. This in turn means increased turnover of NADH molecules. This means that pyruvate will be converted into lactate to a lesser extent and the pH will not drop excessively. It also means that the TCA cycle can continue generating compounds needed for gluconeogenesis, and that the compound glyceraldehyde-3-P may be converted to a lesser extent into glycerol-3-P for fatty acid synthesis. All of these effects play a part in generating more glucose for the brain during alcohol ingestion, thus preventing hangover. By increasing the metabolic rate, there is an increased transfer of NADH into the mitochondrion by the malate-aspartate and the glycerol-phosphate shuttle. This process decreases the level of NADH in the cytosol and thus eliminates the main cause of decreased rate of gluconeogenesis (new glucose formation form amino acids and TCA cycle intermediates), and increased formation of lactic acid. Two main contributing factors of hangover.

Ginger Form Zingiber Officinale.

This compound is known for its effectiveness in the treatment of vertigo (sensation of spinning), nausea, postoperative nausea and vomiting, morning sickness and chemotherapy induced nausea, and motion sickness. All of these symptoms bare striking similarities to the symptoms of hangover. And thus they all may be treated with ginger. Its active constituents are the 6-gingerol and galanolactone. Both of these compounds antagonize (inhibit) the 5-hydroxy-tryptamine type 3 receptors (5-HT3) in the ileum (a part of the small intestine), as well as in the brain in the chemoreceptor trigger zone. When these receptors are stimulated by alcohol they induce the feeling and nausea and may even cause vomiting. By inhibiting these receptors ginger also inhibits these unpleasant sensations commonly sensed during a hangover. Ginger also inhibits the cyclooxygenase, lipoxygenase and thromboxane synthase enzymes. These enzymes produce strong pro-inflammatory compounds.

The B Vitamins.

The B vitamins are the most common vitamins to become deficient during alcohol ingestion. These vitamins also play important roles in the process of gluconeogenesis, the formation of glucose from glycogen, the regeneration of glutathione, and mitigating the formation of lactic acid. They also prevent megaloblastic anemia, and some neurological problems such as the Wernickie-Korsakoff syndrome, parasthesias and paralysis, commonly found in alcoholics. B vitamins combat the depressive effects of ethanol consumption, particularly vitamins B12. Vitamin B6, in addition to combating depression, also assists in the transmission of chemicals in the nervous system. B vitamins are lost during the consumption of ethanol due to the diuretic effect of alcohol.

Vitamin B1 (Thiamine).

Alcoholism is the primary cause of thiamine deficiency in industrialized countries. Beriberi is a clinical manifestation of thiamine deficiency. Cerebral Beriberi may lead to Wemicke's encephalopathy (loss of memory and decreased acquisition of memory) and Korsakoff's Psychosis. Oxidative stress it thought to play an important role in the neurological pathology of Thiamine deficiency. Thiamine takes a part in two very important reactions involved in glucose metabolism. It is a cofactor for pyruvate dehydrogenase complex, and the alpha-ketoglutarate dehydrogenase complex.

These two reactions are necessary for the proper functioning of the TCA cycle which is compromised by the excessive production of NADH. The proper functioning of the TCA cycle is necessary for providing the TCA cycle intermediates necessary for gluconeogenesis. It also keeps glycolysis moving and thus prevents the conversion of pyruvate into lactate (preventing a pH drop) and of glyceraldehyde-3-P into glycerol-3-P (the back bone of fatty acid synthesis). Thiamine is also necessary for the proper functioning of the Pentose-Phosphate pathway. This pathway is necessary for the production of NADPH. This molecule is necessary for reducing oxidized glutathione by the enzyme glutathione reductase. Reduced glutathione removes H2O2 (one of the free radical species) in the reaction catalyzed by glutathione peroxidase. Glutathione is one of the most powerful systems in removing free radicals, and thus it is also one of the most important factors that reduce the symptoms of hangover.

Vitamin B12.

This vitamin is required for normal functioning of the enzyme methionine synthase. This enzyme converts homocysteine to methionine and then to glutathione. Thus this vitamin is necessary for the synthesis of glutathione, the anti-oxidizing system against free radicals. It also plays a part as a coenzyme for the methyl-malonyl-CoA isomerase reaction. This reaction produces succinyl-CoA and then other TCA cycle intermediates which can then be used for gluconeogenesis. Deficiency of this vitamin in addition to causing low levels of glutathione, and producing less intermediates for gluconeogenesis, also causes megaloblastic anemia and neurological problems.

Vitamin B6 (Pyridoxine).

Vitamin B6 functions as a coenzyme for glycogen phosphorylase. This is the enzyme which catalyzes the release of glucose from liver glycogen. It also functions as an anti-oxidizing and anti-inflammatory agent, and has been shown to decrease the number of hangover symptoms by 50% on average in subjects who consumed alcohol.

According to an embodiment of the present invention, there is provided a composition comprising a gingerol, a silymarin, a catechin and a group B vitamin.

In an embodiment of the present invention, which is not meant to be limiting in any manner, the gingerol, preferably from Zingiber officinale radix is present in an amount between about 1 mg and about 125 mg. For example, the composition may comprise a gingerol in an amount of about 1 mg, 2 mg, 5 mg, 10 mg, 25 mg, 50 mg, 75 mg, 100 mg, 120 mg, or about 125 mg. Further, the amount of gingerol present may be defined by a range of any two values listed above.

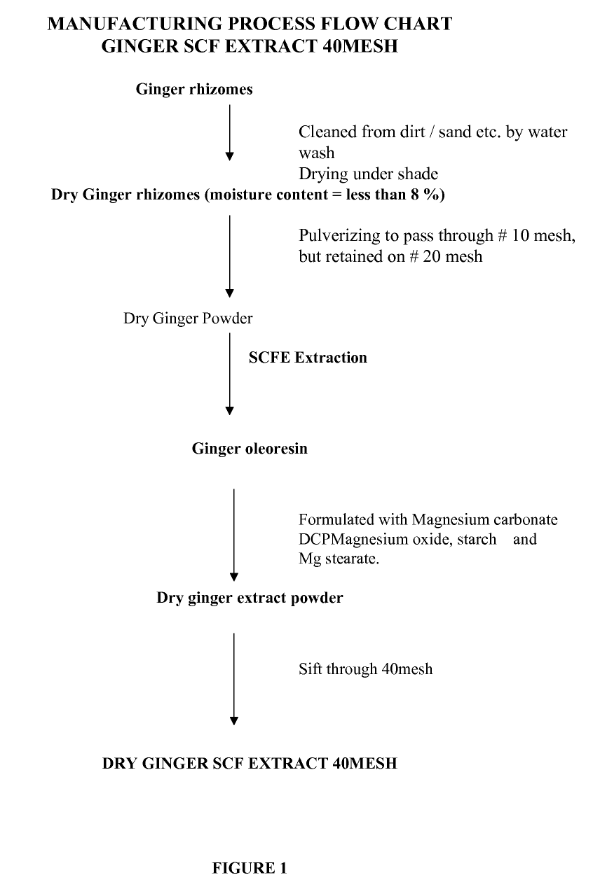

It is contemplated that gingerol may be obtained from an extract of ginger. For example, but not wishing to be limiting in any manner, ginger rhizomes may be subjected to one or more processing steps, such as, but not limited to cleaning, washing for example, but not limited to, water washing or the like, drying, pulvarizing, extraction, for example, but not limited to SCFE extraction, formulating, for example, but not limited to with magnesium carbonate, DCP magnesium oxide, starch, magnesium stearate, or a combination thereof, sifting or the like, for example through a mesh of a particular size, or any combination thereof. In a preferred embodiment, a composition comprising gingerols may be obtained as shown in FIG. 1.

In an embodiment of the present invention, silymarin, preferably from Silybum marianum fructus is present in an amount between about 10 mg and about 630 mg. For example, the composition may comprise silymarin in an amount of about 10 mg, 25 mg, 50 mg, 75 mg, 100 mg, 125 mg, 150 mg, 200 mg, 250 mg, 300 mg, 400 mg, 500 mg, 600 mg, or about 630 mg. Further, the amount of silymarin present may be defined by a range of any two of the values listed above.

Without wishing to be limiting in any manner, silymarin may be provided as an extract of milk thistle. For example, milk thistle fruits may be subjected to one or more processing steps including, but not limited to milling, grinding, grounding, homogenizing, extracting, filtering, concentrating, washing, drying, purifying, using one or more solvents such as but not limited to water, ethyl acetate, alcohols, hexane or a combination thereof.

In an embodiment of the present invention, the catechin present in the composition comprises EGCG, preferably from Camellia sinensis in an amount between about 25 mg and about 800 mg. For example, but without wishing to be limiting in any manner, the composition may comprise EGCG in an amount of about 60 mg, 75 mg, 100 mg, 200 mg, 250 mg, 300 mg, 400 mg, 500 mg, 600 mg, 700 mg, 750 mg, or about 800 mg. Further, the amount of EGCG present may be defined by a range of any two of the values listed above.

Without wishing to be limiting in any manner, EGCG may be obtained as an extract of green tea. For example, green tea leaves may be subjected to one or more processing steps including, but not limited to milling, grinding, grounding, homogenizing, extracting, filtering, concentrating, drying, purifying, using one or more solvents such as but not limited to water, ethyl acetate, alcohols or a combination thereof. Any process of extracting EGCG from green tea known in the art is contemplated herein. In an alternate embodiment, it is possible that green tea may be included in the composition.

In an embodiment of the present invention, the group B vitamin may comprise one or more of vitamin B1, vitamin B6 and vitamin B12. In a preferred embodiment, the composition comprises vitamin B1, vitamin B6 and vitamin B12. When present, vitamin B1 is typically in a range of about 0.75mg to about 250 mg (or higher). For example, the vitamin B1 may be present in an amount of about 0.75 mg, 1 mg, 10 mg, 25 mg, 50 mg, 75 mg, 100 mg, 150 mg, 200 mg, 225 mg or 250 mg. Further, the amount of vitamin B1 present may be defined by a range of any two of the values listed above.

When present, vitamin B6 is typically in a range of about 1 mg to about 250 mg or higher. For example, vitamin B6 may be present in an amount of 1 mg, 10 mg, 25 mg, 50 mg, 75 mg, 100 mg, 150 mg, 200 mg, 225 mg or about 250 mg. Further, the amount of vitamin B6 present may be defined by a range of any two of the values listed above.

When present, vitamin B12 is typically in a range of about 1.5 mcg to about 5000 mcg or higher. For example, vitamin B12 (cyanocobalamin) may be present in an amount of about 1.5 mcg, 5 mcg, 25 mcg, 50 mcg, 100 mcg, 250 mcg, 500 mcg, 750 mcg, 1000 mcg, 1250 mcg, 1500 mcg, 2000 mcg, 2500 mcg, 3000 mcg, 4000 mcg or about 5000 mcg. Further, the amount of vitamin B12 present may be defined by a range of any two of the values listed above.

In an embodiment of the present invention, which is not meant to be limiting, there is provided a composition comprising gingerol, preferably from Zingiber officinale radix in an amount from about 1 mg to about 125 mg; Silymarin, preferably from Silybum marianum fructus in an amount of about 10 mg to about 630 mg; EGCG, preferably from green tea Camellia sinensis in an amount of about 25 mg to about 800 mg; vitamin B1 in an amount of about 0.75 mg to about 250 mg; vitamin B6 in an amount of about 1 mg to about 250 mg and vitamin B12 (Cyanocobalamin) in an amount of about 1.5 mcg to about 5000 mcg.

In a preferred embodiment, which is not meant to be limiting, the composition comprises gingerol, preferably from Zingiber officinale radix in an amount of about 2.5 mg; Silymarin, preferably from Silybum marianum fructus in an amount of about 72 mg; EGCG, preferably from green tea Camellia sinensis in an amount of about 60 mg; vitamin B1 in an amount of about 50 mg; vitamin B6 in an amount of about 125 mg and vitamin B12 (Cyanocobalamin) in an amount of about 400 mcg.

The present invention also contemplates any composition as generally described above, but rather than being defined by a specific quantity of one or more components present, the composition is characterized by the weight ratios of the components present. For example, but not to be considered limiting in any manner, the present invention provides a composition comprising:
Gingerol in an amount of about 0.5% to about 78% (w/w), for example, but not limited to 0.5%, 1%, 5%, 10%, 20%, 25%, 30%, 35%, 40%, 45%, 50%, 55%, 60%, 65%, 70%, 75% or 78% (w/w). Further, the amount of gingerol present may be defined by a range of any two of the values listed above.
Silymarin in an amount of about 0.6% to about 96% (w/w), for example, but not limited to 0.6%, 1%, 2%, 5%, 10%, 20%, 25%, 30%, 40%, 50%, 60%, 70%, 80%, 90%, 95% or 96% (w/w). Further, the amount of silymarin present may be defined by a range of any two of the values listed above.
EGCG in an amount of about 1% to about 98% (w/w), for example, but not limited to 1%, 2%, 5%, 10%, 20%, 25%, 30%, 40%, 50%, 60%, 70%, 80%, 90%, 95%, 96%, 97%, or 98% (w/w). Further, the amount of EGCG present may be defined by a range of any two of the values listed above.
Vitamin B1 in an amount of about 0.04% to about 88% (w/w), for example, but not limited to 0.04%, 0.1%, 0.5%, 1%, 2%, 5%, 10%, 20%, 25%, 30%, 40%, 50%, 60%, 70%, 80%, 85%, 87%, or 88% (w/w). Further the amount of vitamin B1 present may be defined by a range of any two of the values listed above.
Vitamin B6 in an amount of about 0.06% to 88% (w/w), for example, but not limited to 0.06%, 0.1%, 0.5%, 1%, 2%, 5%, 10%, 15%, 20%, 25%, 30%, 40%, 50%, 60%, 70%, 80%, 85%, 87%, or 88% (w/w). Further, the amount may be defined by a range of any two of the values listed above.
Vitamin B12 in an amount of about 7×10⁻⁷% to 12% (w/w), for example, but not limited to 7×10⁻⁶%, 7×10⁻⁵%, 7×10⁻⁴%, 7×10⁻³%, 7×10⁻²%, 7×10⁻¹%, 1%, 2%, 5%, 6%, 7%, 8%, 9%, 10%, 11% or 12% (w/w). Further the amount may be defined by a range of any two of the values listed above.

In a preferred embodiment, the present invention provides a composition comprising:
gingerol in an amount of about 2.5 mg;
Silymarin in an amount of about 72 mg;
EGCG in an amount of about 60 mg;
vitamin B 1 in an amount of about 50 mg;
vitamin B6 in an amount of about 125 mg, and;
vitamin B 12 (Cyanocobalamin) in an amount of about 400 mcg,
   The present invention also contemplates a composition comprising:
gingerol in an amount of about 0.8% (w/w);
Silymarin in an amount of about 23% (w/w);
EGCG in an amount of about 19% (w/w);
vitamin B1 in an amount of about 16% (w/w);
vitamin B6 in an amount of about 40% (w/w) and;
vitamin B12 (Cyanocobalamin) in an amount of about 0.1% (w/w).

Still many other compounds may be used in combination with one or more of the above described compounds in the composition of the present invention. For example but without limitation, ethanol, amino acids, such as methionine or taurine, group C vitamins, simple carbohydrates such as glucose or fructose, fatty acids such as stearic acid, ginseng, gingko biloba, lime extract, minerals, including but not limited to divalent minerals such as calcium and magnesium, methyl sulfonylmethane (MSM), guarana caffeine, licorice root, nicotinic acid, cysteine, cysteic acid, N-acetylcysteine, antacid compounds, one or more salts or any any combination thereof. Any compound which posseses anti-inflammatory and/or antioxidizing activity properties as would be known in the art, may be present in the composition of the present invention.

Compositions may be formulated as desired and a number of inactive ingredients may be added, for example, but not limited to provide longer shelf life, to make the formulation more palatable or presentable, or to decrease manufacturing costs.

In certain examples of the present invention, a composition is for oral administration in a variety of forms, including, without limitation, liquid tincture, beverage, food product such as, but not limited to a health bar or a pudding, lozenge, tablet, caplet, capsule or the like. Pharmaceutically acceptable excipients, solvents, carriers or diluents are well known to the skilled person and may be incorporated as desired. In solid dosage forms for oral administration an active compound(s) may be admixed with an inert excipient such as, but not limited to sodium citrate or dicalcium phosphate; a filler, such as but not limited to starch, lactose, sucrose, glucose, mannitol or silicic acid; a binder, humectant, disintegrating agent, solution retarder, absorption accelerator, wetting agent, absorbent, or any combination thereof as desired.

The daily dosage of compounds may be administered in the form of one or more unit dosage forms, such as a capsule, caplet, tablet or the like. The formulation of an individual dosage unit can be based on the amount of active ingredient(s) that are required to be present in each tablet to total the amount of active ingredient(s) required daily. Extra amounts may be incorporated into the formulation in order to compensate for degradation of active ingredients over time.

There may be more than one source for any given compound. For example, but not to be limiting, EGCG may be provided in its pure form or it may be provided as an extract of green or white tea. In another example, Silymarin may be provided as an extract of Milk Thistle or it may be complexed with compounds such as phosphatidylcholine to improve bioavailability. In still another example, silibinin which is thought to be a major active component of silymarin, is provided.

The present invention also provides a method of preventing, treating or both preventing and treating a negative effect of alcohol consumption in a person. The method comprises administering to such a person a composition comprising a gingerol, a silymarin, a catechin and a group B vitamin. The composition may be administered orally prior to alcohol sonsumption, during alcohol consumption, after alcohol consumption or any combination thereof. In a preferred embodiment, between about 1 and 6, preferably 2 unit doses (i.e. 2 capsules) of the composition as defined in Example 2 is administered per drinking session or per day to a person. In an alternate embodiment, a first dose is administered prior to alcohol consumption, and a second dose is administered after alcohol consumption.

The present invention will be further illustrated in the following examples.

### EXAMPLES

### Example 1

### Preparation of Capsules

The following composition was prepared:
1. Milk Thistle Extract 80% Silymarin (90 mg)
2. Ginger Rhizome Extract 5% Gingerols (50 mg)
3. Green Tea Extract 40% EGCG (150 mg)
4. Vit B 1 as Thiamine HCL (52 mg)
5. Vit B6 as Pyridoxine HCL (158.05 mg)
6. Vit B12 1% (48 mg)
7. Syloid (2.8 mg)
8. Magnesium Stearate (4.2 mg)
9. Flavored Cap (100 mg)

The material was processed in the following manner prior to being packaged in capsules:
A. INTO BLENDER PLACE: (2), (4), (6), PASS (7) THROUGH SCREEN TO REMOVE LARGE PARTICLES, ADD ½ OF (5) AND MIX FOR 7 MINUTES.
B. ADD INTO BLENDER: (1), ½ OF (3), BALANCE OF (5) AND MIX FOR 7 MINUTES.
C. ADD BALANCE OF (3) AND MIX FOR 5 MINUTES.
D. PASS (8) THROUGH SCREEN TO REMOVE LARGE PARTICLES, ADD INTO BLENDER AND MIX FOR 3 MINUTES.
E. DISCHARGE INTO POLYBAG LINED DRUMS, TIE, WEIGHT, LABEL AND SEAL. COMPOSITION IS READY TO BE PACKAGED INTO CAPSULES

Characterization of capsules: Running Weight: 655 mg+ or −5%

Desintegration: As per USP (NMT 45 mins)

### Example 2

Capsules were prepared having the following ingredients in each capsule:

| | | |
|---|---|---|
| Silymarin (from Silybum marianum fructus) | 72 | mg |
| EGCG (from Camellia sinensis folium) | 60 | mg |
| Gingerols (from Zingiber officinale radix) | 2.5 | mg |
| Vitamin B1 (thiamine) | 50 | mg |
| Vitamin B6 (pyridoxine) | 125 | mg |
| Vitamin B12 (cyanocobalamin) | 400 | mcg |

### Example 3

Case Study 1.

Subject woke up with headache, nausea, and lack of energy after a night out drinking and dining. Subject swallowed two capsules as prepared in Example 2 with two glasses of water. Within approximately 20 minutes energy levels were noticeably increased, and the headache and nausea diminished. Subject reported similar satisfying results on at least 3 different occasions. Subject has not experienced any occasion where hangover symptoms were exacerbated by ingesting these capsules.

Case Study 2.

15-20 university students were provided with capsules, prepared according to Example 2, after an all night session of alcohol consumption. Students reported that shortly after ingestion of capsules hangover symptoms were substantially reduced. Furthermore, students reported that simultaneous use of capsules allowed for greater alcohol consumption with less of a feeling of drunkenness.

Case Study 3.

Two professional subjects testified that marathon drinking and dining (heavy drinking on two or more consecutive evenings as may occur during a business trip or conference) was no problem when simultaneously using the capsules as prepared in Example 2. No side effects of the extensive drinking could be felt by these subjects. No headache, no nausea, no lack of energy, no muscle aches and pains was observed. These subjects took one capsule prior to initiating alcohol consumption, and one capsule after alcohol consumption. Depending on the extent of drinking, further capsules were taken in between drinks. Subjects became accustomed to a greater pace of drinking alcohol when simultaneously using the capsules. In the absence of these capsules, subjects were only able to maintain their accustomed pace of drinking alcohol for one evening.

The examples show that compositions comprising a gingerol, a silymarin, a catechin and a group B vitamin can mitigate a negative effect of alcohol consumption. Such compositions not only reduce hangover symptoms but may also address the problem of alcohol-induced organ damage.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of preventing, treating or both preventing and treating a negative effect of alcohol consumption in a person in need of such treatment, which comprises administering to the person a composition comprising a gingerol, a silymarin, a catechin and a group B vitamin.

2. The method of claim 1, wherein said composition is administered orally prior to consuming alcohol or when said negative effect is observed.

3. The method of claim 1, wherein said composition is administered orally prior to alcohol consumption, during alcohol consumption, after alcohol consumption, or any combination thereof.

4. The method of claim 1, wherein said composition is administered orally prior to and after alcohol consumption.

5. The method of claim 1, wherein said negative effect is alcohol-induced hangover.

6. The method of claim 1, wherein said negative effect is alcohol-induced organ damage.

7. The method of claim 1, wherein said negative effect is alcohol-induced drunkenness.

8. The method of claim 1, wherein said gingerol is from Zingiber officinale.

9. The method of claim 1, wherein said silymarin is from Silybum marianum fructus.

10. The method of claim 1, wherein said catechin is epigallocatechin gallate from Camellia sinensis folium.

11. The method of claim 1, wherein said group B vitamin is two or more of Vitamin B 1, Vitamin B6, and Vitamin B 12.

12. A composition for administration to a subject to mitigate a negative effect of alcohol consumption, said composition comprising a gingerol, a silymarin, a catechin, and a group B vitamin.

13. The composition of claim 12, wherein said gingerol is from Zingiber officinale.

14. The composition of claim 12, wherein said silymarin is from Silybum marianum fructus.

15. The composition of claim 12, wherein said catechin is epigallocatechin gallate from Camellia sinensis folium.

16. The composition of claim 12, wherein said group B vitamin is two or more of Vitamin B1, Vitamin B6, and Vitamin B12.

17. The composition of claim 12, wherein said composition is formulated as an oral dosage form.

18. The composition of claim 17, wherein said oral dosage form is selected from the group consisting of: liquid tincture, beverage, food product, lozenge, tablet, caplet, and capsule.

19. The composition of claim 12, wherein said composition is formulated as a capsule, comprising in each capsule:

| | | |
|---|---|---|
| Silymarin (from Silybum marianum fructus) | 72 | mg |
| EGCG (from Camellia sinensis folium) | 32.5 | mg |
| Gingerols (from Zingiber officinale radix) | 2.5 | mg |
| Vitamin B1 (thiamine) | 50 | mg |
| Vitamin B6 (pyridoxine) | 125 | mg |
| Vitamin B12 (cyanocobalamin) | 400 | mcg. |

20. A composition comprising:
Gingerol in an amount of about 0.5% to about 78% (w/w),
Silymarin in an amount of about 0.6% to about 96% (w/w),
EGCG in an amount of about 1% to about 98% (w/w),
Vitamin B1 in an amount of about 0.04% to about 88% (w/w),
Vitamin B6 in an amount of about 0.06% to 88% (w/w), and
Vitamin B12 in an amount of about 7×10⁻⁷% to 12% (w/w).

21. The composition of claim 20, wherein said composition comprises gingerol in an amount of about 0. 1% to about 2% (w/w), silymarin in an amount of about 10% to 30% (w/w), EGCG in an amount of about 10% to 30% (w/w), vitamin B1 in an amount of about 10% to 30% (w/w), vitamin B6 in an amount of about 20% to 50% (w/w) and vitamin B12 (Cyanocobalamin) in an amount of about 0.01% to 1% (w/w).

22. The composition according to claim 21, wherein gingerol is present in an amount of about 0.8% (w/w), silymarin in an amount of about 23% (w/w), EGCG in an amount of about 19% (w/w), vitamin B 1 in an amount of about 16% (w/w), vitamin B6 in an amount of about 40% (w/w) and vitamin B 12 (Cyanocobalamin) in an amount of about 0.1%
